(19) United States
(12) Patent Application Publication
(10) Pub. No.: US 2023/0318003 A1
(43) Pub. Date: Oct. 5, 2023

(54) REDOX FLOW BATTERY AND AQUEOUS-BASED SOLUTION

(71) Applicant: RIVUS AB, MÖLNDAL (SE)

(72) Inventor: Cedrik WIBERG, MÖLNDAL (SE)

(73) Assignee: RIVUS AB, MÖLNDAL (SE)

(21) Appl. No.: 18/021,716

(22) PCT Filed: Aug. 19, 2021
(86) PCT No.: PCT/SE2021/050814

§ 371 Date: Feb. 16, 2023

(30) Foreign Application Priority Data
Aug. 19, 2020 (SE) 2050965-9

(51) Int. Cl. H01M 8/18; H01M 8/08

(52) U.S. Cl. CPC H01M 8/188; H01M 8/08; H01M 2300/0011

(57) ABSTRACT

The present invention relates to a redox flow battery comprising: a positive compartment containing a positive electrode in contact with a first aqueous-based electrolyte solution comprising a positive electrolyte dissolved in a first aqueous-based solvent; and a negative compartment containing a negative electrode in contact with a second aqueous-based electrolyte solution comprising a negative electrolyte being an organic redox-active molecule dissolved in a second aqueous-based solvent wherein at least one of the first and second aqueous-based electrolytes is based on an ammonium-based salt, wherein the organic redox-active molecule is a naphthalene diimide, abbreviated NDI, or a modified NDI.

### TECHNICAL FIELD

The present invention relates to a redox flow battery and an aqueous-based solution.

### BACKGROUND ART

Society faces a large challenge in transitioning to sustainably generating electricity from renewable energy sources such as wind and solar. One of the biggest obstacles to this transition is the intermittence of the sources, leading to a mismatch of supply and demand, a problem most commonly solved by installing large-scale energy storage.

The flow battery has existed for roughly 50 years already, but has been employing metals such as Iron, Chromium and Vanadium, which have serious inherent problems relating to either performance, procurement or toxicity.

Also, organic molecules have been considered for use in flow battery systems, and flow battery systems which are based on different quinones such as anthraquinone and benzoquinone, are known. For example, WO2017170944A1 which relates to an aqueous secondary cell for which at least one of the positive electrode and the negative electrode contains a compound having a naphthalene diimide structure or a perylene diimide structure as an active material. Further, WO18162851A1 which relates to the use of a bis(pyridinium)-naphthalene diimide redox ionic compound used as an active electrode material for an aqueous electrolyte battery.

An energy storage technology, that has the potential to surpass current technology in both performance, cost and environmental benignity, is aqueous organic redox flow batteries. Thus, there is a need for high-performing aqueous organic redox flow battery systems to meet the demands.

### DESCRIPTION OF THE INVENTION

According to a first aspect of the present invention, a redox flow battery is provided.

The redox flow battery comprising:
a positive compartment comprising a positive electrode in contact with a first aqueous-based electrolyte solution comprising a positive electrolyte dissolved in a first aqueous-based solvent;
a negative compartment comprising a negative electrode in contact with a second aqueous-based electrolyte solution comprising a negative electrolyte being an organic redox-active molecule dissolved in a second aqueous-based solvent;
electrical conductive means for establishing electrical conduction between said positive electrode and said negative electrode, and an external load for directing electrical energy into or out of the redox flow battery;
a separator component that separates the first aqueous-based electrolyte solution in the positive compartment from the second aqueous-based electrolyte solution in the negative compartment and substantially prevents the positive electrolyte in the positive compartment and the negative electrolyte in the negative compartment from intermingling with each other, while permitting the passage of non-redox-active species between the electrolyte solutions in the positive and negative compartments;
and means capable of establishing flow of the electrolyte solutions past said positive and negative electrodes, respectively, wherein at least one of the first and second aqueous-based electrolytes is based on an ammonium-based salt, and wherein the organic redox-active molecule is a naphthalene diimide, abbreviated NDI, or a modified NDI.

The positive compartment may be referred to as the positive side of the battery and the negative compartment may be referred to as the negative side of the battery.

According to at least one example embodiment, the positive compartment contains a positive electrode in contact with a first aqueous-based electrolyte solution comprising a positive electrolyte dissolved in a first aqueous-based solvent.

According to at least one example embodiment, the negative compartment contains a negative electrode in contact with a second aqueous-based electrolyte solution comprising a negative electrolyte being an organic redox-active molecule dissolved in a second aqueous-based solvent.

According to at least one example embodiment, the negative electrolyte has a more negative redox potential than the positive electrolyte. According to at least one example embodiment, the negative electrolyte comprises an organic redox-active molecule that has a more negative redox potential than the positive electrolyte, e.g. compared to a corresponding molecule in the positive electrolyte. The difference in reduction potential between the positive electrolyte and the negative electrolyte typically determines the open-circuit voltage.

Further, the redox flow battery also comprises means capable of (i.e., having design features for) establishing flow of the first aqueous-based electrolyte solution and the second aqueous-based electrolyte solution past, or through, said positive and negative electrodes, respectively. Such means may e.g. be a pump or other means resulting in a flow or pressure difference.

During discharge of the redox flow battery, the first aqueous-based electrolyte solution and the second aqueous-based electrolyte solution are, upon operation, pumped through the positive electrode and the negative electrode, respectively, in a fuel cell-like reactor, which involves an electrochemical cell. The negative electrode, for example being a porous electrode, and the positive electrode, e.g. being a porous electrode, are separated by the separator component, e.g. an ion-selective membrane.

Suitable negative electrodes and positive electrodes comprise, e.g. any suitable material/s, for example, woven or non-woven graphite, or other porous carbonaceous materials.

The first and/or second aqueous-based electrolyte solutions may be, for example, water, or water in admixture with water-soluble co-solvent/s and/or supporting electrolytes. Some examples of protic organic solvents, which may be used as a water-soluble co-solvent in the aqueous-based electrolyte solutions, according to the present invention, include the alcohols, such as methanol, ethanol, n-propanol, isopropanol, n-butanol, sec-butanol, isobutanol, t-butanol, n-pentanol, isopentanol, 3-pentanol, neopentyl alcohol, n-hexanol, 2-hexanol, 3-hexanol, 3-methyl-1-pentanol, 3,3-dimethyl-1-butanol, isohexanol, and cyclohexanol. The protic organic solvent, which may be used as a water-soluble co-solvent in the aqueous-based electrolyte solutions, according to the present invention, may alternatively be or include a carboxylic acid, such as acetic acid, propionic acid, butyric acid, or a salt thereof. Some examples of polar aprotic solvents, which may be used as a water-soluble co-solvent in the aqueous-based electrolyte solutions, according to the present invention, include nitrile solvents (e.g., acetonitrile, propionitrile, and butyronitrile), sulfoxide solvents (e.g., dimethyl sulfoxide, ethyl methyl sulfoxide, diethyl sulfoxide, methyl propyl sulfoxide, and ethyl propyl sulfoxide), sulfone solvents (e.g., methyl sulfone, ethyl methyl sulfone, methyl phenyl sulfone, methyl isopropyl sulfone, propyl sulfone, butyl sulfone, tetramethylene sulfone, i.e., sulfolane), amide solvents (e.g., N,N-dimethylformamide, N,N-diethylformamide, acetamide, dimethylacetamide, and N-methylpyrrolidone), ether solvents (e.g., diethyl ether, 1,2-dimethoxyethane, 1,2-diethoxyethane, 1,3-dioxolane, and tetrahydrofuran), carbonate solvents (e.g., propylene carbonate, ethylene carbonate, butylene carbonate, chloroethylene carbonate, fluorocarbonate solvents, dimethyl carbonate, diethyl carbonate, dipropyl carbonate, ethyl methyl carbonate, methyl propyl carbonate, and ethyl propyl carbonate), organochloride solvents (e.g., methylene chloride, chloroform, 1,1,-trichloroethane), ketone solvents (e.g., acetone and 2-butanone), and ester solvents (e.g., 1,4-butyrolactone, ethylacetate, methylpropionate, ethylpropionate, and the formates, such as methyl formate and ethyl formate). The polar aprotic solvent may also be or include, for example, hexamethylphosphoramide (HMPA), 1,3-dimethyl-3,4,5,6-tetrahydro-2(1H)-pyrimidinone (DMPU), or propylene glycol monomethyl ether acetate (PGMEA).

The first and/or second aqueous-based electrolyte solutions may according to at least one example embodiment comprise a supporting electrolyte/s, serving to make the aqueous-based electrolyte solutions ionically conductive and provide the system with mobile charge carriers. According to at least one example embodiment, the supporting electrolyte of at least one of the first and second aqueous-based electrolyte solutions comprises the previously mentioned ammonium-based salt. According to at least one example embodiment, the supporting electrolyte of the other one of the first and second aqueous-based electrolytes (i.e. in embodiments in which only one of the first and second aqueous-based electrolytes is based on the ammonium-based salt) comprises varying concentrations of neat, or mixtures of, solutions of sulfuric acid, hydrobromic acid, chloric acid, perchloric acid, hydrochloric acid, citric acid, carbonic acid, phosphonic acid, phosphoric acid, formic acid, acetic acid, chloride salts of sodium, potassium, magnesium, calcium and ammonium, as well as sodium, potassium, calcium and magnesium salts of carbonate, bicarbonate, phosphate, biphosphate, sulfate, bisulfate, nitrate, citrate, chlorate and perchlorate. According to at least one example embodiment, the supportive electrolyte of both the first and second aqueous-based electrolyte solutions are based on an ammonium-based salt.

According to at least one example embodiment, the second aqueous-based electrolyte solution, or the supporting electrolyte of the same, is based on an ammonium-based salt. According to at least one example embodiment, the first aqueous-based electrolyte solution, or the supporting electrolyte of the same, is not based on an ammonium-based salt.

According to at least one example embodiment, the ammonium-based salt is, or comprises at least, one of the following: ammonium chloride, ammonium phosphate. Additionally or alternatively, the ammonium-based salt is, or comprises at least, one of the following: ammonium sulphate, ammonium nitrate. That is, according to at least one example embodiment, the ammonium-based salt is, or comprises at least, one of the following: ammonium chloride, ammonium phosphate, ammonium sulphate, ammonium nitrate. According to at least one example embodiment, the ammonium-based salt is, or comprises ammonium chloride. According to at least one example embodiment, the ammonium-based salt is, or comprises ammonium phosphate. According to at least one example embodiment, the ammonium-based salt is, or comprises ammonium sulphate. According to at least one example embodiment, the ammonium-based salt is, or comprises ammonium nitrate. When stating that at least one of the first and second aqueous-based electrolytes is based on an ammonium-based salt, the first and/or second aqueous-based electrolytes is formed from the ammonium-based salt, or comprises the ammonium-based salt.

The redox flow battery, according to the present invention, comprises electrical conductive means for establishing electrical conduction between said positive electrode and said negative electrode in order to permit the redox flow battery, according to the present invention, to be charged and discharged. In a particular embodiment, the electrical conduction means include wiring means, i.e., the presence of wiring and associated bonding pads and the like sufficient for establishing electrical conduction. However, the electrical conductive means for establishing electrical conduction do not necessarily have to be in the form of wiring.

For example, electrical conduction may be established by assembling multiple electrodes and multiple flow channels in a stacked bipolar configuration so that electrical connections need only be made to the first and last electrodes.

The redox flow battery, according to the present invention, comprises external load for directing electrical energy into or out of the redox flow battery. For example, the load has a dual function of accepting electrical energy from an electrical source during a charging phase of the redox flow battery, according to the present invention, and also accepting electrical energy from the redox flow battery during a discharging phase of the redox flow battery. According to at least one example embodiment, the load functions only to accept electrical energy from the redox flow battery, according to the present invention, during a discharging phase of the redox flow battery, and a separate source is included for sourcing electrical energy during a charging phase of the redox flow battery. Generally, switching means are included in order to operate the redox flow battery, according to the present invention, in either a charging or discharging mode. For example, when operating in a charging mode, a first switch establishing connection between positive and negative electrodes and an external load is disengaged (i.e., open) while a second switch establishing connection between a source and positive and negative electrodes is engaged (i.e., closed); and in the discharging mode, the first switch establishing circuitry between positive and negative electrodes and an external load is engaged while the second switch establishing circuitry between a source and positive and negative electrodes is disengaged.

In accordance with the present invention the redox flow battery, as described herein, has outstanding voltage and stability. The aqueous-based electrolyte solutions, according to an example embodiment of the present invention, are aqueous-based comprising metal-free electrolytes, and are safe and non-toxic. The organic redox-active molecules, according to the present invention, are manufactured using extremely “green” synthesis routes, including synthesis routes with few steps and one-step synthesis routes.

The redox flow battery, according to the present invention, is thus an especially perfect candidate for large-scale energy storage such as this, also since the production cost typically does not depend on any rare earth metals, like it does for competing technologies. Furthermore, since the redox-active materials, i.e. the aqueous-based electrolyte solutions, may be stored in tanks and pumped through electrochemical cells upon operation, the power/capacity ratio can be scaled up based on what is desired for the specific application.

Apart from the opportunity in storage of solar energy, there are also opportunities for the redox flow battery, according to the present invention, in load levelling, peak shaving, district housing and residential energy storage, wind power, farms, densely populated cities where the cost of installing power transmission is very expensive, amongst others.

Further, the redox flow battery, according an example embodiment of the present invention, also does have the features of enabling decoupled power and capacity, large scale and long term energy storage, that every component is replaceable during maintenance, and/or that it is safe due to the aqueous-based electrolyte solutions.

According to at least one example embodiment, the organic redox-active molecule has a solubility at room temperature of at least 0.4 M in the second aqueous-based electrolyte solution. According to at least one example embodiment, buffer or acid is added to the solution or respective aqueous based electrolyte to increase the solubility.

According to at least one example embodiment, the organic redox-active molecule is a naphthalene diimide, abbreviated NDI, or a modified NDI.

Thus, according to at least one example embodiment the organic redox-active molecule is a modified NDI, or modified naphthalene diimide (NDI) having a solubility at room temperature of at least 0.4 M in the aqueous-based electrolyte solution. According to at least one example embodiment the organic redox-active molecule is a naphthalene diimide (NDI) having a solubility at room temperature of at least 0.4 M in the aqueous-based electrolyte solution.

According to at least one example embodiment all the utilized oxidation states of the organic redox-active molecule is a modified naphthalene diimide (NDI) having a solubility at room temperature of at least 0.4 M in the aqueous-based electrolyte solution.

The solubility of species, the organic redox-active molecule/s, directly influences the energy density of electrolyte solutions, and in extension, battery systems as a whole. A redox flow battery utilizing NDI, or a modified NDI, with a solubility exceeding 0.4 M will offer relatively higher energy densities and will allow higher charge/discharge current densities.

Further, the present invention does also relate to a redox flow battery, as described herein, wherein the organic redox-active molecule is a naphthalene diimide (NDI) and at least one, e.g. one to two, for example one, modified naphthalene diimide (NDI), as described herein, or at least two, e.g. two to three, for example two, modified naphthalene diimide (NDI), as described herein.

According to at least one example embodiment, the aqueous-based electrolyte solution comprises at least two different organic redox-active molecules dissolved in the second aqueous-based solvent, a first organic redox-active molecule being NDI as described above, and a second organic redox-active molecule being, modified naphthalene diimide NDI, as described above.

In further embodiments of the redox flow battery according to the present invention, as described herein, the first aqueous-based solvent and the second aqueous-based solvent are the same.

In further embodiments of the redox flow battery according to the present invention, as described herein, the modified NDI having a solubility at room temperature of at least 0.5 M, at least 0.6 M, at least 0.7 M, at least 0.75 M, at least 0.8 M, at least 0.9 M, or, alternatively, at least 1.0 M, in the aqueous-based electrolyte solution.

In even further embodiments of the redox flow battery according to the present invention, as described herein, the modified NDI has a water solubility at room temperature of at least 0.5 M, at least 0.6 M, at least 0.7 M, at least 0.75 M, at least 0.8 M, at least 0.9 M, or, alternatively, at least 1.0 M.

In further embodiments of the redox flow battery according to the present invention, as described herein, the modified NDI has a solubility of at most 10 M, at most 9.0 M, at most 8.0 M, at most 7.0 M, at most 6.0 M, at most 5.0 M, at most 4.0 M, at most 3.0 M, at most 2.5 M, at most 2.0 M, or, alternatively, at most 1.5 M in the aqueous-based electrolyte solution at room temperature.

In still further embodiments of the redox flow battery according to the present invention, as described herein, the modified NDI has a water solubility at room temperature of at most 10 M, at most 9.0 M, at most 8.0 M, at most 7.0 M, at most 6.0 M, at most 5.0 M, at most 4.0 M, at most 3.0 M, at most 2.5 M, at most 2.0 M, or, alternatively, at most 1.5 M.

In a further embodiment of the present invention, a redox flow battery, as described herein, is disclosed, wherein the modified NDI, is a substituted NDI, e.g. a core-aminated NDI.

According to at least one example embodiment, the modified NDI comprises an amino group. In a further embodiment of the redox flow battery according to the present invention, as described herein, the modified NDI has a structure according to formula I

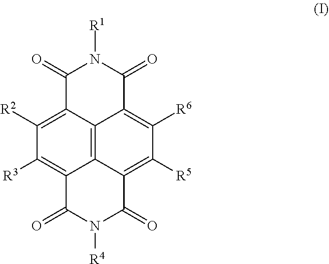

wherein each, of R¹, R², R³, R⁴, R⁵ and R⁶, is independently selected from:
hydrogen atom;
cyano group (—CN);
amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to six carbon atoms, or, R⁸ and R⁹ forming together with the nitrogen a hetero ring having four to six carbon atoms;
sulfonate (—S(O)₂OH or —S(O)₂O⁻) or (—COOH or or COO—);
halogen atoms (e.g. fluorine, chlorine, bromine, and/or iodine); and
group R⁷, being a hydrocarbyl group having one to twenty carbon atoms, and having one or more heteroatoms selected from oxygen, nitrogen, sulfur, and halogen atoms (e.g. fluorine, chlorine, bromine, and/or iodine), or
group R⁷, being a hydrocarbyl group having one to twenty carbon atoms, and being substituted by one, two, or three substituents selected from
   amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to six carbon atoms, or R⁸ and R⁹ forming together with the nitrogen, or together with the nitrogen and a further nitrogen in either of R⁸ or R⁹, a hetero ring having four to six carbon atoms, and
   sulfonate (—S(O)₂OH or —S(O)₂O⁻) or (—COOH or or COO—).

According to at least one example embodiment, R1 and R4 comprises tertiary or quaternary amines, and/or sulfonate groups.

According to at least one example embodiment, at least one of R2, R3, R5 and R6 is a group or molecule comprising more than a hydrogen atom, or is different to a hydrogen atom, e.g. another atom or molecule. That is, at least one of R2, R3, R5 and R6 is not solely hydrogen. This may e.g. improve the aqueous solubility and/or electronic properties of the molecule.

According to at least one example embodiment, the modified NDI, comprises an amino group and/or cyano group on at least one of R2, R3, R5 and R6. R2, R3, R5 and R6 are typically referred to as core substituents or core group, as they are associated with the core of the NDI, or modified NDI, molecule. This may e.g. improve the aqueous solubility and/or electronic properties of the molecule.

According to at least one example embodiment, R2, R3, R5 and R6 comprises at least two amino groups and/or two cyano groups, with hydrogen present on any remaining two core groups. For example, the two amino groups and/or the two cyano groups may be arranged mirrored, i.e. on R2 and R5, or arranged on R3 and R6. This may e.g. improve the aqueous solubility and/or electronic properties of the molecule.

It should be understood that the embodiments related to the chemical structure of the modified NDI above are also applicable to the NDI having a structure according to formula II as described below. In such cases, R21, R22, R23, R24, R25 and R26 correspond to R1 R2, R3, R4, R5 and R6 respectively, wherein R22, R23, R25 and R26 are the core substituents or core groups.

Redox flow batteries utilizing an NDI, or a modified NDI, as described herein, as a redox-active molecule in the electrolyte solution/s offer multiple advantages over analogous aqueous organic redox flow battery systems. In choosing R₁ and R₄, or R₂₁ and R₂₄, as described herein, to be quaternary amines (or tertiary amines which will be protonated upon solution), or sulfonate groups connected to the imide nitrogen by a hydrocarbyl linker, the aqueous solubility of the molecule is promoted, while at the same time two permanent charges are introduced to the structure, without affecting the electronic properties of the NDI, or the modified NDI, as described herein. The permanent charges, apart from giving a solubilizing effect, hinder the NDI, or the modified NDI, as described herein, from crossing over the ion-exchange membrane due to coulombic repulsion.

As seen in FIG. 6 and FIG. 7, continuous cycling at pH 7 resulted in no detectable material degradation, attesting the remarkable chemical stability of the different oxidation states of the molecule. The possibility of employing aqueous solutions at neutral pH infers a significant safety advantage over competing technologies which typically employ concentrated sulfuric acid or hydrobromic acid electrolytes. In the NDI variations containing quaternary amines or sulfonate groups, the species will carry permanent charges resulting in a significantly lowered permeation through the ion-exchange membrane separating the positive aqueous electrolyte compartment from the negative aqueous electrolyte compartment.

As seen in FIGS. 2a, 2b, 8, 9, 10 and 11, the NDI molecules, i.e. the NDI and the modified NDI, as described herein, exhibit more deeply negative reduction potentials than comparative organic flow battery candidates, resulting in an increased battery voltage.

In choosing R², R³, R⁵ and R⁶, or R²², R²³, R²⁵ and R²⁶, as described herein, to be electron-withdrawing or electron-donating groups, the reduction potential of the NDI and the modified NDI, as described herein, can be tuned for the specific application, potentially maximizing the operating voltage of the battery. Due to the less negative reduction potentials of the modified NDI, it can be used in conjunction with NDI, resulting in a battery system that has a fast-charge part, characterized by the reduction of the modified NDI, suitable for when there is demand for high power output, and a slow-charge part, characterized by the reduction of NDI, suitable for long-term energy storage.

In still a further embodiment of the redox flow battery according to the present invention, as described herein, the modified NDI has the structure according to formula I, and wherein each, of R¹, R², R³, R⁴, R⁵ and R⁶, is independently selected from:
hydrogen atom;
cyano group (—CN);
amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to six carbon atoms, or, R⁸ and R⁹ forming together with the nitrogen a hetero ring having four to six carbon atoms;
sulfonate (—S(O)₂OH or —S(O)₂O⁻);
halogen atoms (e.g. fluorine and/or bromine); and
group R⁷, being a hydrocarbyl group having one to sixteen carbon atoms, and having one or more, e.g. one, two, or three, for example, one or two, heteroatoms selected from nitrogen, sulfur, and halogen atoms (e.g. fluorine and/or bromine), or
group R⁷, being a hydrocarbyl group having one to sixteen carbon atoms, and being substituted by one or two, substituents selected from
   amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to six carbon atoms, or R⁸ and R⁹ forming together with the nitrogen, or together with the nitrogen and a further nitrogen in either of R⁸ or R⁹, a hetero ring having four to six carbon atoms, and sulfonate (—S(O)₂OH or —S(O)₂O⁻).

In even a further embodiment of the redox flow battery according to the present invention, as described herein, the modified NDI has the structure according to formula I, wherein, three or less, of R¹, R², R³, R⁴, R⁵ and R⁶, are hydrogen.

In a further embodiment of the redox flow battery according to the present invention, as described herein, the modified NDI has the structure according to formula I, wherein R² and R⁵ are both hydrogen.

In still a further embodiment of the redox flow battery according to the present invention, as described herein, the modified NDI has the structure according to formula I, wherein each, of R¹, R³, R⁴ and R⁶, is independently selected from:
hydroxy group (—OH);
methoxy group (—MeOH);
cyano group (—CN);
amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to four carbon atoms, or, R⁸ and R⁹ forming together a hetero ring having four to six carbon atoms;
sulfonate (—S(O)₂OH or —S(O)₂O⁻);
halogen atoms (e.g., fluorine and/or bromine); and
group R⁷, being a hydrocarbyl group having one to ten carbon atoms, and having one or more heteroatoms selected from nitrogen, sulfur, and halogen atoms (e.g., fluorine and/or bromine), or
group R⁷, being a hydrocarbyl group having one to ten carbon atoms, and being substituted by one or two substituents selected from
   amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to four carbon atoms, or R⁸ and R⁹ forming together with one or two nitrogens a hetero ring having four to six carbon atoms; and
      sulfonate (—S(O)₂OH or —S(O)₂O⁻).

In even a further embodiment of the redox flow battery according to the present invention, as described herein, the modified NDI has the structure according to formula I, wherein each, of R¹, R³, R⁴ and R⁶, is independently selected from:
cyano group (—CN);
amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to four carbon atoms, or, R⁸ and R⁹ forming together a hetero ring having four to six carbon atoms;
sulfonate (—S(O)₂OH or —S(O)₂O⁻);
halogen atoms (e.g., fluorine and/or bromine); and
group R⁷, being a hydrocarbyl group having one to ten carbon atoms, and having one or more heteroatoms selected from nitrogen, sulfur, and halogen atoms (e.g., fluorine and/or bromine), or
group R⁷, being a hydrocarbyl group having one to ten carbon atoms, and being substituted by one or two substituents selected from
   amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to four carbon atoms, or R⁸ and R⁹ forming together with one or two nitrogens a hetero ring having four to six carbon atoms; and sulfonate (—S(O)₂OH or —S(O)₂O⁻).

In still a further embodiment of the redox flow battery according to the present invention, as described herein, the modified NDI has the structure according to formula I, wherein each, of R³ and R⁶, is independently selected from:
hydrogen;
cyano group (—CN);
amino group —NR⁸R⁹R¹⁰, wherein R¹⁰ is present when the amino group is quaternized, and wherein R⁸, R⁹ and R¹⁰, if R¹⁰ present, are independently selected from hydrogen atom and a hydrocarbyl group R¹¹ having one or two carbon atoms.
bromine or iodine.

In even a further embodiment of the redox flow battery according to the present invention, as described herein, the modified NDI has the structure according to formula I, wherein each, of R¹ and R⁴, is independently selected from:
hydrogen;
ethyl sulfonate; and
group R⁷, being a hydrocarbyl group having two to four carbon atoms, and being substituted by one or two substituents selected from
   amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to two carbon atoms.

According to at least one example embodiment, the battery is configured such that the NDI, or modified NDI, is reduced with two electrons in the negative compartment, creating reducedNDl in the form of an NDI dianion, NDI⁻ or hydroNDI, NDIH₂. Hereby, an improved battery is provided. According to at least one example embodiment, the reduced NDI is an original reduced NDI having a first structure, and the battery is configured such that the original reduced NDI is restructured into a restructured reduced NDI having a second structure different from said first structure, the restructured reduced NDI having a different reduction potential compared to the original reduced NDI. The difference in reduction potential between the original reduced NDI and the restructured reduced NDI may determine the voltage of the battery.

Such configuration of the battery may e.g. comprise adapting the pH of the electrolyte. For example, the pH at the negative compartment may be lower compared to the positive compartment with at least a value of pH 2. Such configuration may additionally or alternatively comprise changing electrolyte (e.g. NaCl instead of KCl), setting suitable temperature, and/or adapting the substituents of the NDI molecule.

According to at least one example embodiment, the pH is adjusted during cycling due to the proton-coupled electron transfer of NDI.

Thus, depending on pH and substituent of the NDI, the reduction may include the reaction of NDI+2e⁻+2H⁺→NDIH₂. Here, as two protons are assimilated, pH will increase automatically, in response to a well balanced concentration of NDI and buffer capacity of the solution.

In embodiments of the present invention, a redox flow battery, as described herein, is disclosed, wherein also the positive electrolyte is a NDI, e.g. the same as the negative electrolyte, or a redox flow battery, as described herein, is disclosed, wherein also the positive electrolyte is an organic redox-active molecule being a modified NDI, as described herein, e.g. the same as the negative electrolyte.

In other words, according to at least one example embodiment, the positive electrolyte is the same as the negative electrolyte, forming a symmetrical redox flow battery.

In embodiments of the present invention, a redox flow battery, as described herein, is disclosed, wherein also the positive electrolyte is an organic redox-active molecule being NDI or a modified NDI as described herein.

In further embodiments of the present invention, a redox flow battery, as described herein, is disclosed, wherein the positive electrolyte and the negative electrolyte are the same modified NDI as described herein.

In the embodiments of the present invention, where a redox flow battery, as described herein, is disclosed, wherein also the positive electrolyte is a NDI, e.g. the same as the negative electrolyte, or a redox flow battery, as described herein, is disclosed, wherein also the positive electrolyte is an organic redox-active molecule being a modified NDI, as described herein, e.g. the same as the negative electrolyte, the cost of production for the NDI and/or the modified NDI will be drastically reduced, due to the double scale. Further, in keeping the amount of different substances in the electrolyte solutions to a minimum, the range of possible degradation mechanisms are also decreased. Further, in utilizing the same molecule, i.e. the same organic redox-active molecule, as both the negative and positive redox-active material, any capacity decay due to species permeating the membrane will be reversible through electrolyte remixing, greatly enhancing the cycling lifetime of the system.

According to at least one example embodiment of the symmetrical redox flow battery, charging of the battery results in the following reactions:
in the negative compartment: NDI+2e⁻→NDI²⁻ and NDI²⁻→NDI*²⁻, wherein NDI*²⁻ is the restructured NDI dianion described above;
in the positive compartment: NDI*²⁻→NDI*⁺2e⁻ and NDI*→NDI, wherein NDI* is the oxidised condition of the restructured NDI dianion.

According to at least one example embodiment of the symmetrical redox flow battery, charging of the battery results in the following reactions:
in the negative compartment: NDI+2e⁻+2H⁺→NDIH₂ and NDIH₂→NDIH₂*, wherein NDIH₂* is the restructured hydroNDI described above;
in the positive compartment: NDIH₂*→NDI*+2⁻+2H⁺ and NDI*→NDI, wherein NDI* is the oxidised condition of the restructured hydroNDI, NDIH₂*.

According to a second aspect of the present invention, an aqueous solution is provided. The aqeuous solution comprises the NDI, or modified NDI, as described in relation to the first aspect of the invention, and an aqueous-based electrolyte based on an ammonium-based salt, e.g. ammonium chloride or ammonium phosphate. The ammonium salt is preferably making up a supporting electrolyte in a redox flow battery.

According to at least one example embodiment, the ammonium-based salt is, or comprises at least, one of the following: ammonium chloride, ammonium phosphate. Additionally, or alternatively, the ammonium-based salt is, or comprises at least, one of the following: ammonium sulphate, ammonium nitrate.

In the aqueous solution, the modified NDI may have a structure according to formula

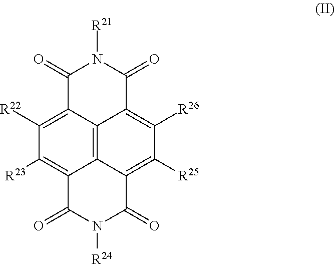

wherein R²² and R²⁵ are both hydrogen atom, and each, of R²¹, R²³, R²⁴ and R²⁶, is independently selected from:
hydrogen atom (H);
cyano group (—CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or, R²⁸ and R²⁹ forming together with the nitrogen a hetero ring having four to six carbon atoms;
sulfonate (—S(O)₂OH or —S(O)₂O⁻) or (—COOH or or COO—);
halogen atoms (e.g. fluorine and/or bromine); and
group R²⁷, being a hydrocarbyl group having one to six carbon atoms, and having one or more heteroatoms selected from nitrogen, sulfur, and halogen atoms (e.g. fluorine and/or bromine), or
group R²⁷, being a hydrocarbyl group having one to six carbon atoms, and being substituted by one or two substituents selected from:
   cyano group (—CN);
   amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or R²⁸ and R²⁹ forming together with the nitrogen, or together with the nitrogen and a further nitrogen in either of R²⁸ or R²⁹, a hetero ring having four to six carbon atoms; and
   sulfonate (—S(O)₂OH or —S(O)₂O⁻) or (—COOH or or COO—).

The modified NDI as described herein, e.g. the modified NDI having a structure according to formula II, has a high chemical stability, easy and green synthesis, high aqueous solubility and attractive electronic properties. This makes the modified NDI valuable for use in various aqueous electrochemical applications such as electrocatalysis or sensoring.

In a further embodiment of the present invention, the modified NDI, as described herein, is disclosed, wherein each of R²¹ and R²⁴, is independently selected from:
group R²⁷, being a hydrocarbyl group having one to six, e.g. one to four, carbon atoms, and having one or more, e.g. one or two, heteroatoms selected from nitrogen, sulfur, and halogen atoms (e.g. fluorine and/or bromine), or
group R²⁷, being a hydrocarbyl group having one to six, e.g. one to four, carbon atoms, and being substituted by one or two, e.g. one, substituents selected from:
   cyano group (—CN);
   amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or R²⁸ and R²⁹ forming together with the nitrogen a hetero ring having four to six carbon atoms; and
   sulfonate (—S(O)₂OH or —S(O)₂O⁻), and
      wherein R²³ and R²⁶, is independently selected from:
cyano group (CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or, R²⁸ and R²⁹ forming together with the nitrogen a hetero ring having four to six carbon atoms;
sulfonate (—S(O)₂OH or —S(O)₂O⁻);
halogen atoms (e.g. fluorine and/or bromine); and
group R²⁷, being a hydrocarbyl group having one to six carbon atoms, and having one or more heteroatoms selected from nitrogen, sulfur, and halogen atoms (e.g. fluorine and/or bromine), or
group R²⁷, being a hydrocarbyl group having one to six carbon atoms, and being substituted by one or two substituents selected from:
   cyano group (—CN);
   amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or R²⁸ and R²⁹ forming together with the nitrogen a hetero ring having four to six carbon atoms; and
   sulfonate (—S(O)₂OH or —S(O)₂O⁻).

A further embodiment of the modified NDI according to the present invention, as described herein, is provided wherein the modified NDI has the structure according to formula II, and wherein R²³ and R²⁶, is independently selected from:
cyano group (CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or, R²⁸ and R²⁹ forming together with the nitrogen a hetero ring having four to six carbon atoms;
sulfonate (—S(O)₂OH or —S(O)₂O⁻); and
group R²⁷, being a hydrocarbyl group having one to six carbon atoms, and having one or more heteroatoms selected from nitrogen, sulfur, and halogen atoms (e.g. fluorine and/or bromine), or
group R²⁷, being a hydrocarbyl group having one to six carbon atoms, and being substituted by one or two substituents selected from:
   cyano group (—CN);
   amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or
   R²⁸ and R²⁹ forming together with the nitrogen a hetero ring having four to six carbon atoms; and
   sulfonate (—S(O)₂OH or —S(O)₂O⁻).

An even further embodiment of the modified NDI according to the present invention, as described herein, is provided wherein the modified NDI has the structure according to formula II, and wherein R²¹ and R²⁴, is independently selected from:
cyano group (CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or, alternatively, R²⁸ and R²⁹ forming together with the nitrogen a hetero ring having four to six carbon atoms;
sulfonate (—S(O)₂OH or —S(O)₂O⁻); and
group R²⁷, being a hydrocarbyl group having one to six carbon atoms, and having one or more heteroatoms selected from nitrogen, sulfur, and halogen atoms (e.g. fluorine and/or bromine), or
group R²⁷, being a hydrocarbyl group having one to six carbon atoms, and being substituted by one or two substituents selected from:
   cyano group (—CN);
   amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³⁰ having one to four carbon atoms, or R²⁸ and R²⁹ forming together with the nitrogen a hetero ring having four to six carbon atoms; and
   sulfonate (—S(O)₂OH or —S(O)₂O⁻).

A further embodiment of the modified NDI according to the present invention, as described herein, is provided wherein the modified NDI has the structure according to formula II, and each, of R²¹ and R²⁴, is independently selected from:
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four, e.g. one to three, for example, one or two, carbon atoms, or R²⁸ and R²⁹ forming together with the nitrogen a hetero ring having four to six carbon atoms;
sulfonate (—S(O)₂OH or —S(O)₂O⁻);
group R²⁷, being a hydrocarbyl group having one to six, e.g. one to three, carbon atoms, and having one or more, e.g. one to three, for example, one or two, heteroatoms selected from nitrogen and sulfur, or
group R²⁷, being a hydrocarbyl group having one to six carbon atoms, e.g. one to three, and being substituted by one or two substituents selected from:
   amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, e.g. one to three, for example, one or two, or R²⁸ and R²⁹ forming together with the nitrogen a hetero ring having four to six carbon atoms; and
   sulfonate (—S(O)₂OH or —S(O)₂O⁻);
      and each, of R²³ and R²⁶, is independently selected from:
cyano group (CN);
amino group —NR²⁸R²⁹, wherein R²⁸ and R²⁹ are independently selected from hydrogen atom and hydrocarbyl group R³¹ having one to four, e.g. one to three, for example, one or two, carbon atoms, or, alternatively, R²⁸ and R²⁹ forming together with the nitrogen a hetero ring having four to six carbon atoms;
sulfonate ( —S(O)₂OH or —S(O)₂O⁻);
group R²⁷, being a hydrocarbyl group having one to six, e.g. one to three, carbon atoms, and having one or more, e.g. one to three, for example, one or two, heteroatoms selected from nitrogen and sulfur, or
group R²⁷, being a hydrocarbyl group having one to six carbon atoms, e.g. one to three, and being substituted by one or two substituents selected from:
   cyano group (—CN);
   amino group —NR²⁸R²⁹, wherein R²⁸ and R²⁹ are independently selected from hydrogen atom and hydrocarbyl group R³¹ having one to four carbon atoms, e.g. one to three, for example, one or two, or, alternatively, R²⁸ and R²⁹ forming together with the nitrogen a hetero ring having four to six carbon atoms; and
   sulfonate (—S(O)₂OH or —S(O)₂O⁻).

In still a further embodiment of the modified NDI according to the present invention, as described herein, the modified NDI has the structure according to formula II, wherein R²¹ and R²⁴ are, e.g. the same, selected from:
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four, e.g. one to three, for example, one or two, carbon atoms;
sulfonate (—S(O)₂OH or —S(O)₂O⁻);
group R²⁷, being a hydrocarbyl group having one to six, e.g. one to three, carbon atoms, and having one or more, e.g. one to three, for example, one or two, heteroatoms selected from nitrogen and sulfur, or
group R²⁷, being a hydrocarbyl group having one to six carbon atoms, e.g. one to three, and being substituted by one or two substituents selected from:
   amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, e.g. one to three, for example, one or two; and
   sulfonate (—S(o)₂OH or —S(O)₂O⁻).

In a further embodiment of the modified NDI according to the present invention, as described herein, the modified NDI has the structure according to formula II, wherein each, of R²³ and R²⁶, is independently selected from:
hydrogen;
cyano group (—CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and a hydrocarbyl group R³¹ having one or two carbon atoms.
bromine or iodine;

In still a further embodiment of the modified NDI according to the present invention, as described herein, the modified NDI has the structure according to formula II, wherein each, of R²¹ and R²⁴, is independently selected from:
hydrogen;
ethyl sulfonate (—C2H4S(O)2OH or —C2H4S(O)2O⁻);
group R²⁷, being a hydrocarbyl group having two to four carbon atoms, and being substituted by one or two substituents selected from:
   amino group —NR2²⁸R2²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to two carbon atoms.

In further embodiments of the redox flow battery, according to the present invention, as described herein, the modified NDI has the structure according to formula II, as described herein. That is, according to a third aspect of the present invention, a redox flow battery according to the first aspect of the invention, with an aqueous solution according to the second aspect of the invention is provided.

In a further embodiment according to the present invention, a redox flow battery, as described herein, is disclosed wherein the modified NDI has a structure according to formula II, as described herein, wherein R²² and R²⁵ are both hydrogen atom, R²¹ and R²⁴ are both a hydrocarbyl group having one to six, for example one to three, e.g. three carbon atoms, each hydrocarbyl group substituted by one amino group —NR²⁸R²⁹R³⁰, wherein R³⁰ is present when the amino group is quaternized, and wherein R²⁸ and R²⁹ are both a hydrocarbyl group having one to four carbon atoms, for example one to three, e.g. one, carbon atoms, and R³⁰, if R³⁰ present, is a hydrogen or a hydrocarbyl group having one to four carbon atoms, for example one to three, e.g. one, carbon atoms and

R²³ and R²⁶ are both an amino group —NR²⁸R²⁹, wherein R²⁸ and R²⁹ are both a hydrocarbyl group having one to four carbon atoms, for example one to three carbon atoms, e.g. one carbon atom.

In a still further embodiment according to the present invention, a redox flow battery, as described herein, is disclosed wherein the modified NDI is the free base or the protonated amine form of either:
4,9-Dibromo-2,7-bis[3-(dimethylamino)propyl]benzo[Imn][3,8]phenanthroline-1,3,6,8(2H,7H)-tetrone, or
4,9-bis(dimethylamino)-2,7-bis(3-(dimethylamino)propyl)benzo[Imn][3,8]phenanthroline-1,3,6,8(2H,7H)-tetraone.

In a further embodiment according to the present invention, a redox flow battery, as described herein, is disclosed wherein the modified NDI has a structure according to formula II, as described herein, wherein R²² and R²⁵ are both hydrogen atom, R²¹ and R²⁴ are both a hydrocarbyl group having one to six, for example one to three, e.g. three carbon atoms, each hydrocarbyl group substituted by one amino group —NR²⁸R²⁹R³⁰, wherein R³⁰ is present when the amino group is quaternized, and wherein R²⁸ and R²⁹ are both a hydrocarbyl group having one to four carbon atoms, for example one to three, e.g. one, carbon atoms, and R³⁰, if R³⁰ present, is a hydrogen or a hydrocarbyl group having one to four carbon atoms, for example one to three, e.g. one, carbon atoms and R²³ and R²⁶ are both cyano group (CN) or, alternatively, R²³ and R²⁶ are both sulfonate (—S(O)₂OH or —S(O)₂O⁻).

According to at least one example embodiment, the sulfonate group (—S(O)₂OH or —S(O)₂O⁻), for NDI or modified NDI, may be replaced with a carboxylate group (—COOH or or CO)—), applicable to both the first and second aspects of the invention.

The present invention further relates to a method for producing a redox flow battery as described herein.

The present invention also relates to a method for producing a modified NDI having the structure according to formula II as described herein.

### BRIEF DESCRIPTION OF DRAWINGS

The invention will be described in greater detail in the following, with reference to the embodiments that are shownin the attached drawings, in which:

FIG. 1 shows a titration curve of 50 ml 15.6 mM tertiary NDI in water by addition of 1 M sodium hydroxide.

FIGS. 2a and 2b show cyclic voltammograms (CVs) for 1 mM NDI in a) neutral phosphate buffer and b) 1 M H₂SO₄.

FIG. 3 shows Potential-pH diagram of NDI. At pH below 3, the second reduction follows a one electron-1.5 proton slope (in total 2 electrons and three protons), and at higher pHs, it is independent of pH, as the first reduction couple is over the whole range.

FIG. 4 shows rotating disk electrode (RDE) analysis for 1 mM NDI in 1 M H₂SO₄.

FIG. 5 shows RDE for NDI in pH 7 phosphate buffer solution at concentrations of a), b) 1 mM and c), d) 50 mM.

FIG. 6 shows bulk electrolytic cycling of 25 mM NDI in pH 6.4 phosphate buffer with a phosphate total concentration of 0.5 M.

FIG. 7 shows cyclic voltammograms comparing the redox activity of the solution before and after 12 cycles in the bulk electrolysis cell.

FIG. 8 shows Cyclic voltammograms of “the compound obtained in Example 3” measured versus Ag/AgCl: in 1 M H₂SO₄ at different scan rates.

FIG. 9 shows Cyclic voltammograms of “the compound obtained in Example 3” measured versus Ag/AgCl: in 0.5 M sodium phosphate buffer pH 7 at different scan rates.

FIG. 10 shows Cyclic voltammograms of “the compound obtained in Example 4b” measured versus Ag/AgCl: in 1 M H₂SO₄ at different scan rates.

FIG. 11 shows Cyclic voltammograms of “the compound obtained in Example 4b” measured versus Ag/AgCl: in 0.5 M sodium phosphate buffer pH 7 at different scan rates.

FIG. 12 shows a schematic view of an aqueous quinone-based redox flow battery.

FIG. 13 shows a cyclic voltammograms of 15 mM partially reduced NDI in 1 M sulfuric acid solution. Sweep rate=100 mV/s.

FIG. 14 shows bulk electrolysis cycling of 15 mM NDI in 1 M sulfuric acid solution.

FIG. 15 shows the capacity utilization for four different redox flow batteries based on different NDIs in combination with different aqueous based electrolytes.

### DETAILED DESCRIPTION

The embodiments of the invention with further developments described in the following are to be regarded only as examples and are in no way intended to limit the scope of the protection provided by the patent claims.

### EXPERIMENTAL SECTION

### EXAMPLES

### Example 1

### Naphthalene Diimide (NDI)

NDI was synthesized in 30 g scale according to Sissi, C. et al, Bioorg. Med. Chem. 2007, 15, (1), 555-62, in a facile and green manner with high yields and water as the only byproduct, and the quaternization, if necessary, simply by bubbling chloromethane through a chloroform solution of the tertiary NDI, whereupon the pure product is precipitated, see Scheme.

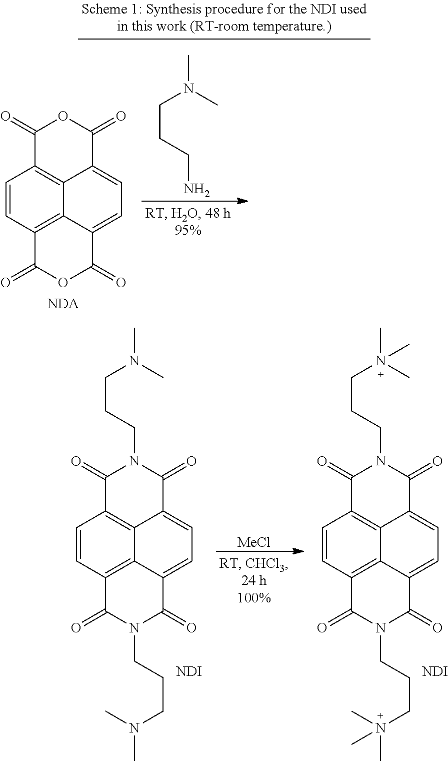

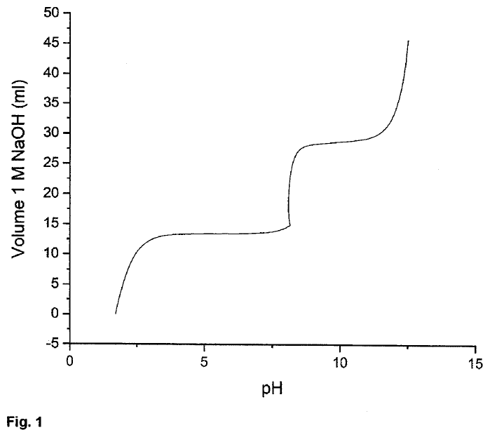

The quaternization is only necessary if the molecule is to be examined at higher pH values, since the tertiary amine sidechains are protonated for pH values less than about 8 (pKa≈8.1) as shown by the titration curve in FIG. 1. In FIG. 1 it can be seen that the molecule deprotonates above pH 8 and precipitates out of solution. For actual flow battery applications, i.e. redox flow battery according to the present invention, the last reaction step is thus possible to omit.

### Electrochmistry

### Cyclic Voltammetry (CV)

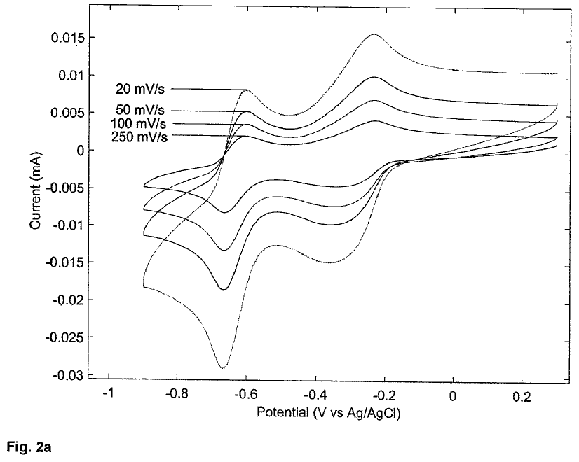

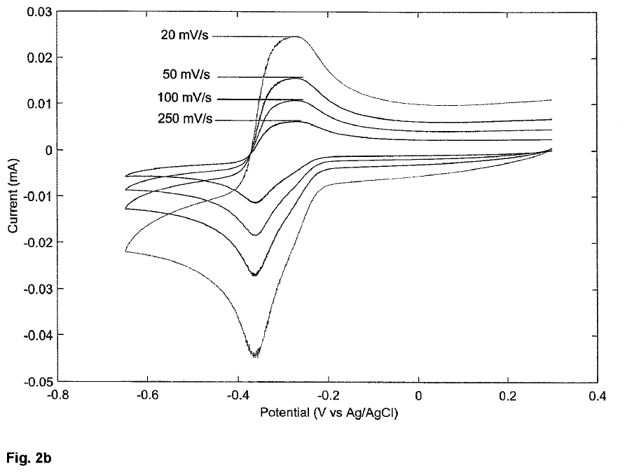

The electrochemistry of NDI was examined by CV at a range of different pH values, to gain understanding of the electron transfer reaction mechanism. FIG. 2a and FIG. 2b show cyclic voltammograms (CVs) for NDI at pH 7 and 0, respectively. An initial observation shows that more than one electron is involved, due to the broadness of the peak at pH 0, and due to the occurrence of two separate redox couples at pH 7. The broadness of the first reduction at pH 7, is assumed to be due to the possibility for the first electron to add at different positions of NDI.

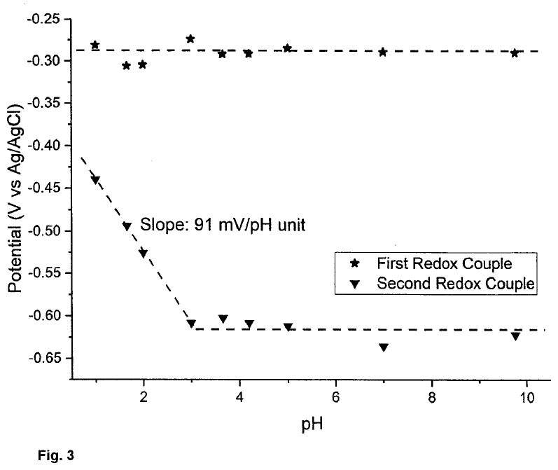

A plot of the reduction potentials for NDI at different pHs is seen in FIG. 3. The stars show that the first reduction potential is independent of pH and does not involve any protons. The second reduction, the triangles in the figure, has a slope of 91 mV/pH at pH below 3, indicating a one electron-1.5 proton relationship, according to the Nernst equation.

It should be remarked that the reduction potential of NDI is outstandingly low, and if used in a redox flow battery, it would possibly yield an operating voltage among the highest of the previously reported structures in aqueous solutions. Indeed, more negative potentials are not beneficial, due to the parasitic hydrogen evolution reactions eventually taking precedence.

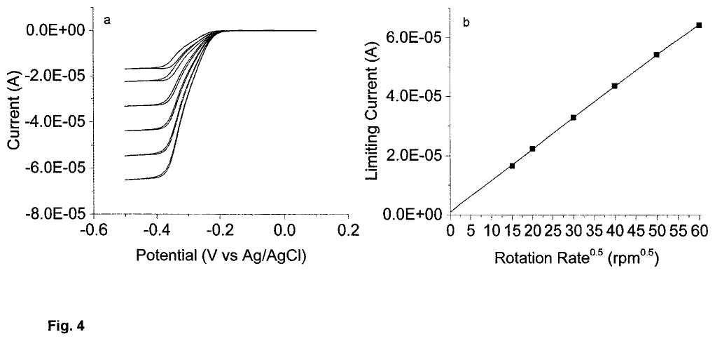

Further, rotating disk electrode (RDE) voltammetry was coupled with diffusion NMR to assess the accessible concentration of NDI in a neutral buffered solution, and tested whether it corresponded to dimerization. The diffusion coefficients from diffusion NMR were found to be 2.57 and 2.34×10⁻⁶ cm² s⁻¹ for the acid and neutral solutions respectively. From the slope in FIG. 4, the accessible concentration was calculated, through the Levich equation, and showed that the reduction of 1 mM NDI at pH 0 only involves two electrons. Addition of three protons to each NDI molecule at pH values as high as 3 is unlikely, since the third proton would be at a weakly basic carbonyl. This is further supported by the lack of buffering at lower pHs in the titration curve in FIG. 1.

Based on this relationship and reasoning, the reduction scheme in Scheme 2 is proposed for acidic solutions as follows.

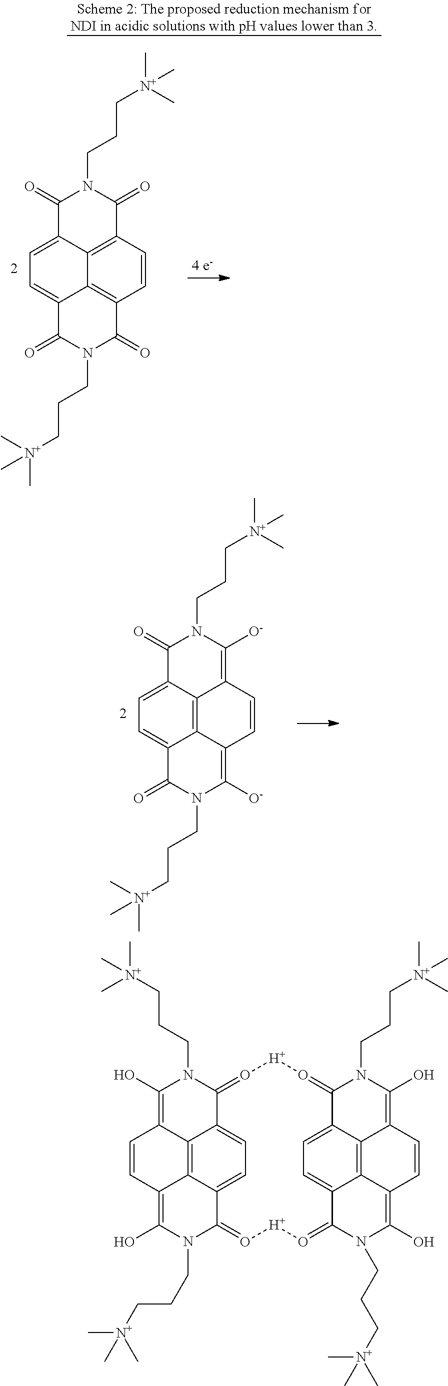

### Dimerization

As work on 9,10-Anthraquinone-2,7-disulfonic acid (AQDS) has shown the impact of dimerization on its electrochemical properties, it was prudent to examine whether the same behavior could be seen for NDI. Since NDI has a large aromatic core, and it is known to self-associate quite readily, it was imagined that the effect could be quite strong, despite the flexible positively charged sidechains.

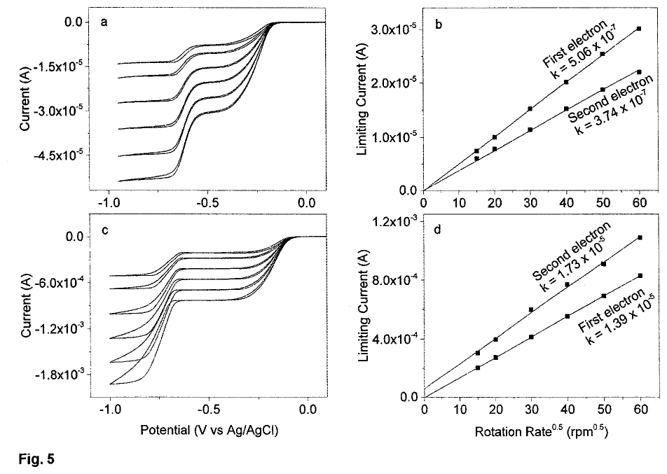

FIG. 5 shows the results from RDE measurements of NDI in neutral buffered solution. Two well-defined diffusion-limited current plateaus were observed, with the first reduction coming at a less negative potential, and the second reduction at a more negative potential, for 50 mM compared to 1 mM. The large distance between reduction potentials could enable the use of NDI in a symmetric flow battery, where NDI is utilized as both cathodic and anodic electrolytes simultaneously, but has not been further examined up to this point. The diffusion-limited currents were fitted to the Levich equation along with the diffusion coefficient acquired from diffusion NMR, showing a linear relationship. The slope of the fit showed, through the Levich equation, that the full capacity is not accessed on the voltammetry timescale, possibly due to dimerization. However, as seen in the next section, the full capacity can be accessed through bulk electrolysis, and it is thought that the dimer dissociates too fast to impede the redox reaction in the flow battery application.

### Bulk Electrolysis

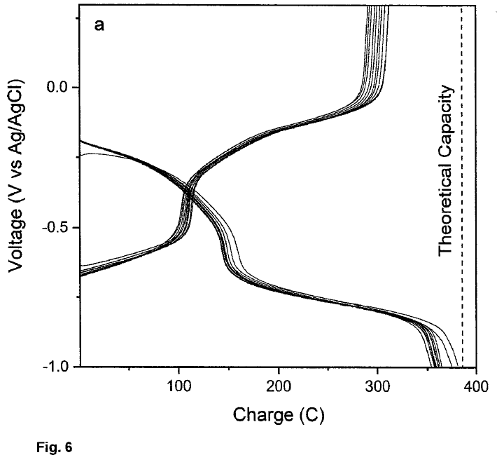

A solution of 25 mM NDI in pH 6.4 sodium phosphate buffer was cycled in a bulk electrolysis cell, where all of the material is reduced, as opposed to CV and RDE, where only a small fraction of the material is probed. A galvanostatic BE setting was used, where a constant current of 40 mA was applied. The electrolysis was intermittently paused, allowing for CVs to be collected to be able to quantify the amount of NDI that has been reduced. The peak heights from the CVs were correlated to the passed current, and it was seen that the complete capacity of NDI could be accessed using bulk electrolysis. See here FIG. 6 which shows bulk electrolytic cycling of 25 mM NDI in pH 6.4 phosphate buffer with a phosphate total concentration of 0.5 M. A possible explanation to why NDI can be quantitatively reduced with BE, and AQDS cannot, is the formation of the AQDS quinhydrone upon reduction, where the reduced species associates with the pristine molecule, to form a new self-association complex, which seems to, similarly to the AQDS dimer, impede the reduction. For NDI, the reduction of the molecule lowers the monomer concentration in the solution, and the dimerization equilibrium is shifted in the direction of dimer separation. Two plateaus were seen at the electrolysis, corresponding to the two separate redox couples, illustrated by the overlaid CV in FIG. 6.

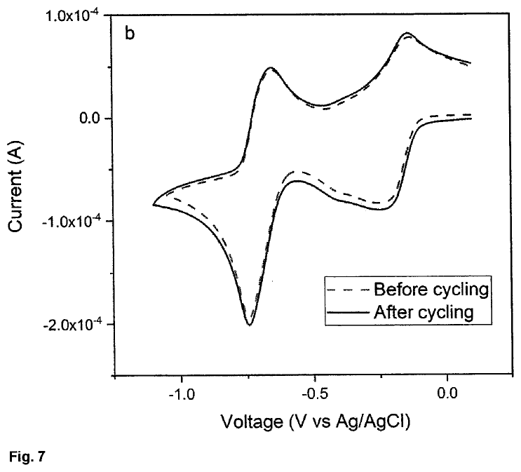

After the initial sweep, continuous cycling at a current of 15 mA was performed for twelve cycles. A CV was collected after the cycling had finished, roughly a week after starting. The CV was compared to the initial, untouched solution, and it was seen that they overlapped perfectly, indicating no or little material degradation over the course of the experiment, showing the exceptional stability of NDI. See here FIG. 7 which shows cyclic voltammograms comparing the redox activity of the solution before and after 12 cycles in the bulk electrolysis cell. Thus, bulk electrolysis shows (see FIG. 6 and FIG. 7) that NDI does not degrade at all, after being cycled 12 times over a week's time. Apart from that, the NDI molecule is synthesized via a one-step reaction, at room temperature in water with an almost 100% yield. It is water soluble up to 0.7 M, and has a lower reduction potential than any of the other examined molecules, resulting in a high battery voltage in the application.

The cost and performance of the aqueous redox flow battery depends very strongly on the redox-active material employed. The starting material for the synthesis of NDI, 1,4,5,8-naphthalene tetracarboxylic acid dianhyride is easily synthezied from naphthalene—a very cheap and abundant compound. The combination of having a very low reduction potential, high aqueous solubility, high chemical stability, low membrane permeability, environmental benignity, possibility for use at neutral pH, and potential for manufacture at very low costs, is a set of criteria that is not easily fulfilled. NDI achieves a fulfillment of these criteria to a far higher extent than competing technologies. Thus, a flow battery employing NDI has a superior cost-performance relationship than competing technologies, without compromising on environmental impact or operational safety.

### Example 2

### 2,6-dibromonaphtalene-1,4,5,8-tetracarboxylic dianhydride

A 250 ml round-bottom flask charged with 1,4,5,8-naphthalene tetracarboxylic acid dianhydride (NDA) (4.00 g, 14.92 mmol) and sulfuric acid (98%, 100 ml) was subjected to stirring at 50° C. for an hour to enhance dissolution. To the resulting solution was added by portions dibromoisocyanuric acid (DBI) (6.46 g, 22.51 mmol) and stirred at ambient temperature for 30 minutes. The reaction temperature was then adjusted and maintained at 120° C. for 45 hours.

Upon heating, reddish-brown fumes evolved which were trapped in a solution of sodium thiosulphate. The reaction mixture was cooled to room temperature, remaining reddish fumes were removed with a gentle stream of nitrogen gas and then poured into crushed ice. The precipitate formed was filtered, washed with copious amount of distilled water and then methanol. Br,Br-NDA was obtained as a yellowish-green solid and vacuum-dried at 40° C. overnight to afford approximately 78% yield. This was used without any further purification.

The nuclear magnetic resonance (NMR) spectroscopy for 2,6-dibromonaphtalene-1,4,5,8-tetracarboxylic dianhydride is: ¹H NMR (400 MHz, DMSO-d6) δ 8.77 (s, 2H); ¹³C NMR (101 MHz, DMSO-d6) δ 158.39, 156.89, 137.97, 129.87, 127.85, 124.72, 123.87.

### Example 3

### 4,9-dibromo-2,7-bis(3-(dimethylamino)propyl)benzo[Imn][3,8]phenanthroline-1,3,6,8(2H,7H)-tetraone

2,6-dibromonaphtalene-1,4,5,8-tetracarboxylic dianhydride from Example 2 (5.01 g, 11.76 mmol) and 50 ml of glacial acetic acid were charged into a two-necked round-bottom flask under stirring. To the system was added 3-(dimethylamino)-1-propylamine (3.61 g, 4.45 ml, 35.33 mmol, 3 eq). The mixture was stirred at 120° C., and after 30 minutes of reaction, cooled to room temperature, quenched in ice, neutralized with sodium carbonate and extracted three times with chloroform. The organic layer was dried under vacuum to give an orange crude, which was purified by column chromatography (CHCl3:MeOH, 9:1) to obtain approximately 20% yield. The silica stationary phase was pretreated with triethylamine.

The nuclear magnetic resonance (NMR) spectroscopy for 4,9-dibromo-2,7-bis(3-(dimethylamino)propyl)benzo[Imn][3,8]phenanthroline-1,3,6,8(2H,7H)-tetraone is: ¹H NMR (400 MHz, Chloroform-d) δ 8.98 (s, 2H), 4.31-4.22 (m, 4H), 2.45 (t, J=7.0 Hz, 4H), 2.24 (s, 12H), 1.99-1.85 (m, 4H); ¹³C NMR (101 MHz, Chloroform-d) δ 160.79, 160.75, 138.99, 128.28, 127.70, 125.32, 124.10, 57.06, 45.23, 39.90, 25.54.

### Example 4a and 4b

### Benzo[Imn][3,8]phenanthroline-2,7-dipropanaminium, 1,3,6,8-tetrahydro-N,N,N,N′,N′,N′-hexamethyl-1,3,6,8-tetraoxo-, dichloride (Example 4a) and 3,3′-(4,9-dibromo-1,3,6,8-tetraoxo-1,3,6,8-tetrahydrobenzo[Imn][3,8]phenanthroline-2,7-diyl)bis(N,N,N-trimethylpropan-1-aminium) chloride (Example 4b)

A 100 ml round-bottom flask was charged at each reaction with 50 mg of either NDI or Br,Br-NDI for the synthesis of compound of Example 4a and compound of Example 4b, respectively. To the system was added 10-15 ml of chloroform and subjected to heating in an oil bath at 50° C. while stirring. Chloromethane gas was bubbled through the content of the flask three times for at most a minute each time. Precipitates were observed to form after an hour of stirring. The system was left to stir for 12 hours. The reaction was cooled to room temperature, filtered and dried under vacuum to afford a quantitative yield.

The compound of Example 4a was obtained as an off-white solid, and the nuclear magnetic resonance (NMR) spectroscopy for Benzo[Imn][3,8]phenanthroline-2,7-dipropanaminium, 1,3,6,8-tetrahydro-N, N, N,N′,N′, N′-hexamethyl-1,3,6,8-tetraoxo-, dichloride is:

¹H NMR (400 MHz, Deuterium Oxide) δ 8.41 (s, 4H), 4.09 (t, J=7.0 Hz, 4H), 3.44-3.35 (m, 4H), 3.02 (s, 18H), 2.20-2.07 (m, 4H); ¹³C NMR (101 MHz, Deuterium Oxide) δ 163.88, 130.96, 125.86, 125.81, 64.00, 52.88, 37.63, 21.34.

The compound of Example 4b was obtained as an orange-red solid and the nuclear magnetic resonance (NMR) spectroscopy for 3,3′-(4,9-dibromo-1,3,6,8-tetraoxo-1,3,6,8-tetrahydrobenzo[Imn][3,8]phenanthroline-2,7-diyl)bis(N,N,N-trimethylpropan-1-aminium) chloride is:

¹H NMR (400 MHz, Deuterium Oxide) δ 8.56 (s, 2H), 4.07 (t, J=7.0 Hz, 4H), 3.44-3.36 (m, 4H), 3.02 (s, 18H), 2.15-2.06 (m, 4H). ¹³C NMR (101 MHz, Deuterium Oxide) δ 161.21, 138.48, 128.22, 126.52, 124.31, 123.31, 63.85, 63.82, 63.79, 55.15, 52.95, 52.91, 52.87, 42.74, 38.33, 21.14.

### Solubility Testing

A saturated solution of about 500 μl each of the targeted molecules, i.e the compounds obtained in Examples 3, 4a and 4b, in milli-Q water containing undissolved particles were prepared at ambient temperature. The solutions were centrifuged at 12,500 rpm for 30 minutes. 100 μl of the supernatant were pipetted in triplicates from each of the solution into separate empty vials of known weights. The content of these vails were left to dry in a vacuum oven overnight. The final weights of the vials were determined and the amount of each of the compounds calculated from the weight difference. Solubility in mmol/L for each compound was finally determined. The results obtained are presented in Table 1, where “Compound 1” is the compound obtained in Example 3, “Compound 2” is the compound obtained in Example 4a and “Compound 3” is the compound obtained in Example 4b. It should be noted that the solubility may be increased by adding a buffer or acid. For example, by adding a buffer or acid to compound 1, the solubility is increased above 0.4 M.

TABLE 1

Solubility (mmol/L)

| Compound | Test 1 | Test 2 | Test 3 | Average |
|---|---|---|---|---|
| 1 | 35.97 | 34.06 | 35.59 | 35.21 |
| 2 | 653.42 | 689.14 | 691.00 | 677.85 |
| 3 | 142.39 | 151.02 | 154.18 | 149.20 |

Solubility of the compound obtained in Example 3 which is a base NDI is lower than those of the compounds obtained in Examples 4a and 4b which are quaternary ammonium NDI salts. This is due to the differences in intermolecular interactions of the individual compounds. The compound obtained in Example 3 contains soluble amines both at the imide positions and the naphthalene core eventually affects the solubility at the molecular level. Adversely, a long range intermolecular π-π stacking effect of the molecules in solution could not be hindered, therefore limiting their solubility. Since the compounds obtained in Examples 4a and 4b exist as salts, they could dissolve more easily in water due to electrostatic interactions of their resulting ions. This could limit the π-π stacking of the NDI to an extent of permitting higher solubilities than compound 1. The difference in the solubilities of the compounds obtained in Examples 4a and 4b is resulting from the insoluble bromine substituent on the naphthalene core. If the structure obtained in Example 3 was to be added to a slightly acidic solution, the amines would become protonated and the solubility would increase significantly. The structures of the compounds obtained in Examples 3, 4a and 4b are shown in Scheme 3.

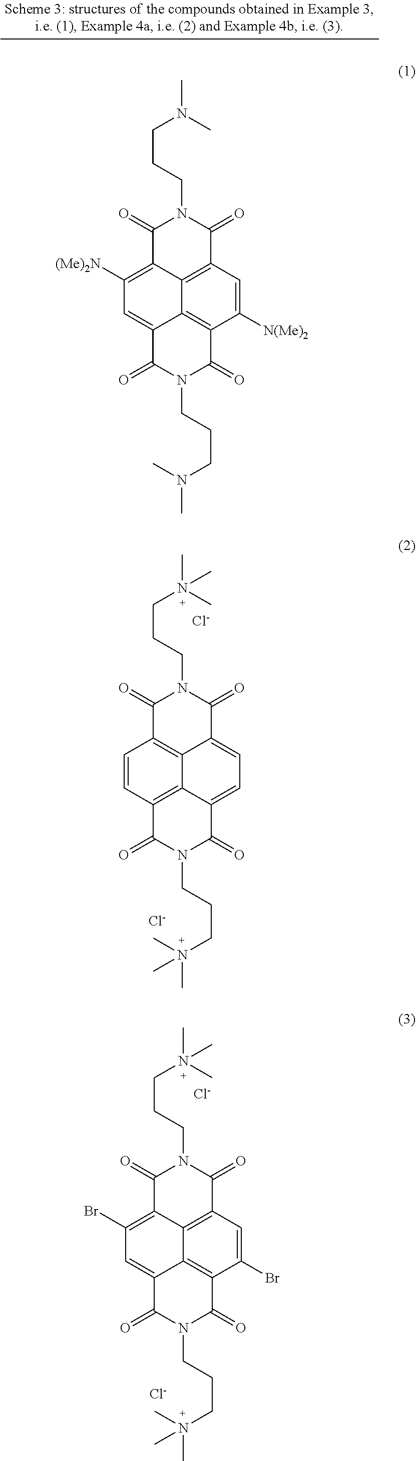

### Electrochemical Measurements

All CV measurements were carried out in a custom built 10 ml three-electrode electrochemical cell. The lower part of the cell holds the electrolyte of interest. In the upper part are ports to hold the working electrode, reference electrode and auxiliary electrode as well as inlet and outlet for N₂ gas. 1 mM solutions of compounds, i.e the compounds obtained in Examples 3, 4a and 4b, each in either 1 M sulfuric acid or 0.5 M sodium phosphate buffer of pH 7 electrolytes. Cyclic voltammograms were recorded at scan rates of 10 mVs⁻¹, 20 mVs⁻¹, 50 mVs⁻¹, 100 mVs⁻¹ and 250 mVs⁻¹ at room temperature. Prior to measurements, the electrolyte was de-aerated by continuously purging with N₂ gas for 10-15 minutes and maintaining a N₂ flow-blanket throughout the experiment to minimize any environmental contamination. Also, 80% of the ohmic drop was compensated for during the experiment using a positive feedback-loop.

### CV Analysis

### The Compound Obtained in Example 3

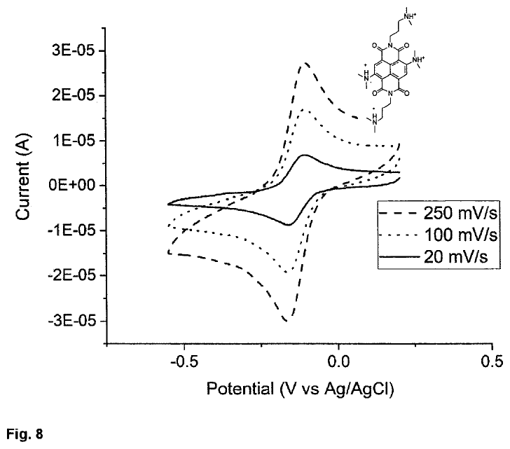

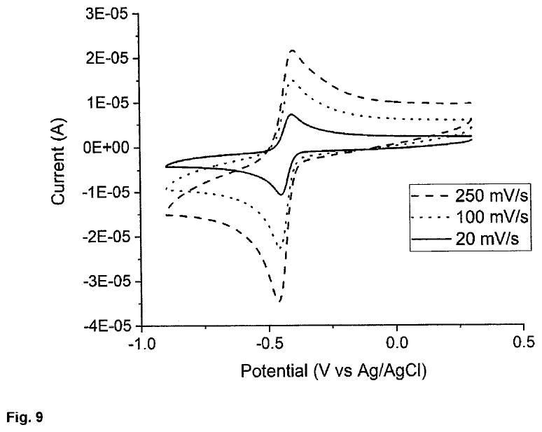

FIG. 8 and FIG. 9 provides CV reports of “the compound obtained in Example 3” in acidic and neutral electrolytes at different scan rates showing single reduction and reversible oxidation peaks. Corresponding numerical data are as summarized in Tables 2 and 3. The redox potential, E^{0′}, obtained was −0.13 V vs. Ag/AgCl in the acidic electrolyte and −0.43 V vs. Ag/AgCl in the neutral electrolyte. At scan rates of 10 mVs⁻¹, 20 mVs⁻¹ and 50 mVs⁻¹, the compound revealed comparatively low peak-to-peak potential separations, ΔEₚ, in the neutral electrolyte than in the acidic electrolyte. Nonetheless, variation of ΔEₚ with increasing scan rate was more pronounce in the neutral electrolyte than in the acidic electrolyte as shown in the respective tables. The results are suggestive that “the compound obtained in Example 3” is generally electrochemically reversible although the AEp are observed to drift with increasing scan rates. Electrochemical reversibility of this compound was further established by calculating peak current ratios, iₚₐ/i_{pc}, at the various scan rates as shown in the Tables 2 and 3. For an ideal reversible process, this parameter should be unity. The values as shown in the tables indicate that “the compound obtained in Example 3” exhibited good reversibility in the acidic electrolyte than in the neutral electrolyte. The varying CV data in both electrolytes predict that the redox chemistry of this compound can be altered by the choice of electrolyte.

TABLE 2
CV data of “the compound obtained in Example 3” in
1M sulfuric acid at different scan rates

| Scan Rate (mVs⁻¹) | E^{0′} vs. Ag/AgCl (V) | ΔEₚ (mV) | $\frac{{i}_{a}}{{i}_{c}}$ |
|---|---|---|---|
| 10 | −0.13 | 63.67 ± 10.03 | 0.91 ± 0.05 |
| 20 | −0.13 | 63.31 ± 10.97 | 0.92 ± 0.01 |
| 50 | −0.13 | 64.97 ± 10.46 | 0.89 ± 0.02 |
| 100 | −0.13 | 66.31 ± 9.29 | 0.87 ± 0.00 |
| 250 | −0.13 | 71.25 ± 11.85 | 0.82 ± 0.01 |

TABLE 3
CV data of “the compound obtained in Example 3” in 0.5M
sodium phosphate buffer of pH 7 at different scan rates

| Scan Rate (mVs⁻¹) | E^{0′} vs. Ag/AgCl (V) | ΔEₚ (mV) | $\frac{{i}_{a}}{{i}_{c}}$ |
|---|---|---|---|
| 10 | −0.43 | 49.21 ± 8.49 | 0.82 ± 0.04 |
| 20 | −0.43 | 51.87 ± 9.16 | 0.80 ± 0.05 |
| 50 | −0.43 | 61.48 ±12.09 | 0.74 ± 0.06 |
| 100 | −0.43 | 68.59 ± 14.05 | 0.69 ± 0.07 |
| 250 | −0.43 | 86.49 ± 20.99 | 0.63 ± 0.07 |

### The Compounds Obtained in Examples 4a and 4b

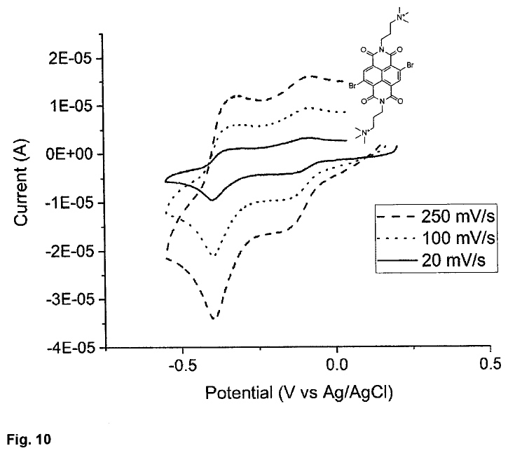

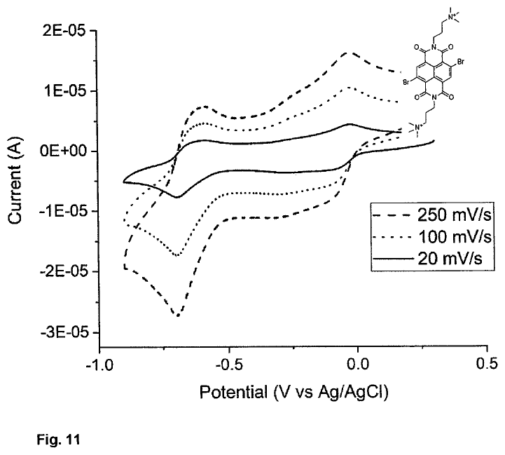

The CVs for the compounds obtained in Examples 4a and 4b revealed two pseudo-reversible redox processes in both the acidic and neutral electrolytes, see CVs for the compounds obtained in Example 4b displayed in FIG. 10 and FIG. 11. The shapes of the CV waves do not give a clear separation between the different redox processes thus, transfer from one complete redox process to the other as the potential ramps does not show a good exponential decay of the peak currents which makes evaluation of the individual processes appear complex. There was no evidence of decomposition observed for these compounds in both electrolytes. The results can be suggestive that the compounds exhibit sluggish redox chemistries in the chosen supporting electrolytes.

The CVs of “the compound obtained in Example 4a” in the acidic electrolyte at 1 mM concentration appeared to give a single but broad reduction and reverse oxidation peaks (CVs not shown). Further investigations were conducted by running CVs for 10 mM and 25 mM concentration of the compound in the acid electrolyte. These then revealed CV waves with two pairs of peaks almost merged together which is characteristic of two separate electron transfer steps with similar redox potentials. When “the compound obtained in Example 4a” was examined in the neutral electrolyte, the resolution of these pairs of peaks become more pronounced (CVs not shown). In the acidic electrolyte, the redox potential, E^{0′}, was approximated to be −0.32 V vs. Ag/AgCl. In the neutral electrolyte, the two observed redox processes could be approximated to occur at potentials, E^{0′}, −0.30 V vs. Ag/AgCl and −0.63 V vs. Ag/AgCl.

The compound obtained in Example 4b gave the most complicated CVs. Two observable pairs of peaks appeared at approximated potentials −0.13 V vs. Ag/AgCl and −0.38 V vs. Ag/AgCl in the acidic electrolyte. In the neutral electrolyte, the redox processes were observed at −0.05 V vs. Ag/AgCl and −0.65 V vs. Ag/AgCl. The broad cathodic and anodic peaks appearing respectively in the first and second redox processes coupled with the high capacitance made it difficult to evaluate peak-to-peak potential separations. Although the voltammograms are complex the systems are in total reversible.

Electrochemical Behaviour of the Target NDIs i.e. “Compound Obtained in Example 3”, “Compound Obtained in Example 4a”, and “Compound Obtained in Example 4b”

Different electrochemical behaviours were observed for the synthesized NDIs i.e. “compound obtained in Example 3”, “compound obtained in Example 4a”, and “compound obtained in Example 4b” in the acidic and neutral electrolytes. The redox potential of each compound showed dependency on pH. All compounds except for “the compound obtained in Example 3” displayed two redox waves characteristic of two separate electron transfer processes. “The compound obtained in Example 3” showing only one redox wave may be that the two-electron transfer processes occur simultaneously at similar potentials at a relatively fast rate. Effects of the different NDI core-substituents and the different background electrolytes can be seen by the shift of the redox potentials. Although the second redox processes for “compounds obtained in Examples 4a and 4b” appear to occur at the same potential, it is clear that the influences of the core substitutions are pronounced on the first redox process. The potential gap between the first and second redox waves narrows down by pushing the former wave to a more negative redox potential when moving from the electron withdrawing bromo-substituents on “compound obtained in Example 4b” to the electron donating amino-groups on “compound obtained in Example 3”. By comparing the compounds in the acidic electrolytes (red CV waves), “the compound obtained in Example 3” shows a redox potential which is more positive than that of “the compound obtained in Example 4a” but almost at the same potential as the first redox wave of the “compound obtained in Example 4b”. This is because in the acidic electrolyte, the amino substituents at the core of “the compound obtained in Example 3” are protonated causing a switch from their usual electron donating properties to electron withdrawing and eventually leading to a facile shift of the redox wave to a more positive potential than that of “the compound obtained in Example 4a”. In the neutral electrolyte (blue CV waves), the potential gap between the first and second redox processes for “the compounds obtained in Examples 4a and 4b” becomes relatively wider compared to their respective behaviours in the acidic electrolyte. The electron withdrawing bromo-substituents on “the compound obtained in Example 4b” influences the first redox wave to appear at a more positive potential than that of “the compound obtained in Example 4a” as a result of an inductive effect of the bromides.

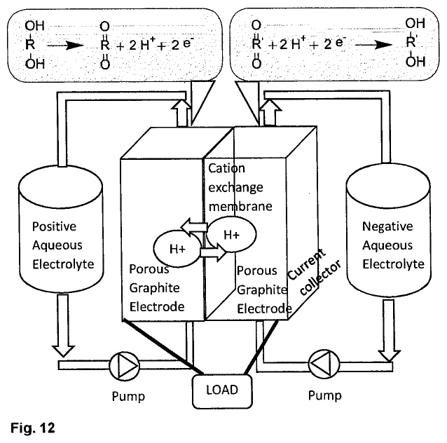

FIG. 12 shows a schematic view of an aqueous quinone-based redox flow battery.

Further, in FIG. 12 the electrolyte solutions in the two tanks contain two different (redox-active molecules) RMs, and the difference in reduction potential between the two molecules determines the open-circuit voltage. The electrolyte solution containing the RM with the more negative redox potential is called the negative electrolyte, and the solution containing the RM with the less negative (or positive) redox potential is called positive electrolyte. A supporting electrolyte serves to make the solution ionically conductive and provide the system with mobile charge carriers.

During discharge, the electrolyte solutions are pumped through an electrochemical cell consisting of two porous electrodes, which are separated by an ion-selective membrane. The molecules in the positive electrolyte, once they reach the surface of the porous electrode, get oxidized and give off electrons which are conducted through an external circuit and used as electricity. At the same time, the negative electrolyte receives electrons from the porous electrode on the cathodic side, and thus gets reduced. Since electrons have effectively been transported from one side of the cell to the other, a charge imbalance has arisen. To negate this, cation or a proton migrates through the membrane from the anodic (positive) to the cathodic (negative) chamber of the electrochemical cell. Thus, FIG. 12 shows the operation of a quinone-based organic flow battery.

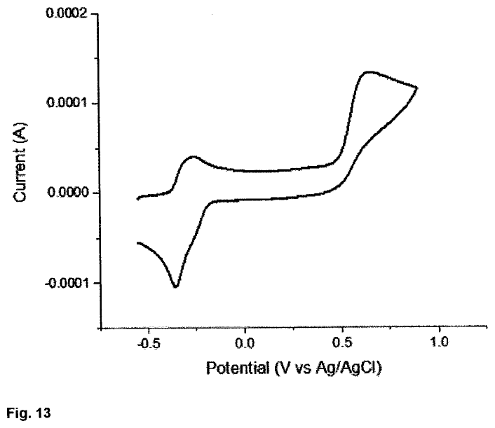

FIG. 13 shows a cyclic voltammogram of 15 mM partially reduced NDI in a 1 M sulfuric acid solution. The figure shows a large separation between multiple two-electron redox processes, enabling the system for use in a symmetrical flow battery setup. In more details, the symmetrical redox flow battery comprises a negative electrolyte with an NDI molecule (e.g. NDI or modified NDI as previously described) which is reduced with two electrons on the negative side (i.e. in the negative compartment), creating the reduced NDI in the form of the NDI dianion, NDI²⁻ or hydroNDI, NDIH₂. The symmetrical redox flow battery is configured such that the reduced NDI restructure, e.g. chemically restructure, into a restructured reduced NDI: NDI*₂₋, having a different structure different to the reduced NDI (NDI²⁻). The restructured reduced NDI (NDI*²⁻) has a different reduction potential from the reduced NDI (NDI²⁻) and is oxidized at relatively higher potentials than the reduced NDI (NDI²⁻). The difference in reduction potential between the reduced NDI (NDI²⁻) and the restructured reduced NDI (NDI*²⁻) determines the voltage of the battery. The main advantage of the NDI restructuring mechanism is that a twice as high number of electrons is accessible for battery utilization. The following reactions are believe to occur:
on the negative side (in the negative compartment): NDI+2e⁻→NDI²⁻ and NDI²⁻→NDI*²⁻, wherein NDI*²⁻ is the restructured reduced NDI (NDI anion) described above;
on the positive side (in the positive compartment): NDI*²⁻→NDI*+2e⁻ and NDI*→NDI, wherein NDI* is the oxidised condition of the restructured redcued NDI.

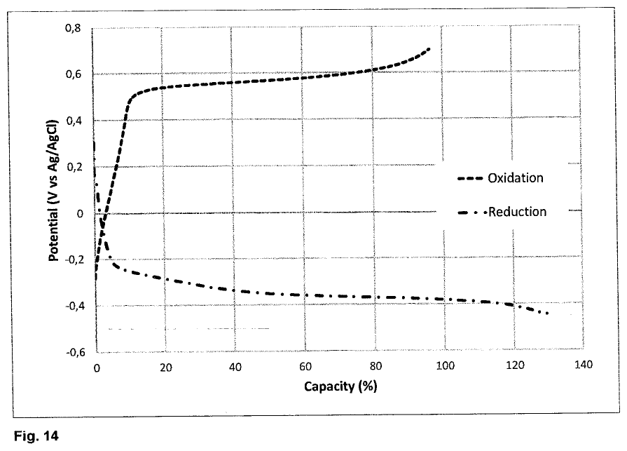

FIG. 14 shows a polarization curve of a bulk electrolysis cell containing 15 mM NDI in 1 M sulfuric acid. In more details, the symmetrical redox flow battery comprises a negative electrolyte with an NDI molecule (e.g. NDI or modified NDI as previously described) which is reduced with two electrons on the negative side (i.e. in the negative compartment), creating the hydroNDI species, NDIH₂. The symmetrical redox flow battery is configured such that hydroNDI, NDIH₂, restructures, e.g. chemically restructures, into a restructured hydroNDI, NDIH₂*, having a different structure different to hydroNDI, NDIH₂. The restructured hydroNDI, NDIH₂*, has a different reduction potential from the to hydroNDI, NDIH₂ and is oxidized at relatively higher potentials than hydroNDI, NDIH₂. The difference in reduction potential between the original hydroNDI, NDIH₂ and the restructured hydroNDI, NDIH₂* determines the voltage of the battery. The main advantage of the NDI restructuring mechanism is that a twice as high number of electrons is accessible for battery utilization. The following reactions are believed to occur:
on the negative side (in the negative compartment): NDI+2e⁻+2H⁺→NDIH₂ and NDIH₂→NDIH₂*, wherein NDIH₂* is the restructured hydroNDI described above;
on the positive side (in the positive compartment): NDIH₂*→NDI*+2e⁻+2H⁺ and NDI*→NDI, wherein NDI* is the oxidised condition of the restructured hydroNDI, NDIH₂*.

The NDI may e.g. be dissolved in an acidic electrolyte, for example 1 M sulfuric acid.

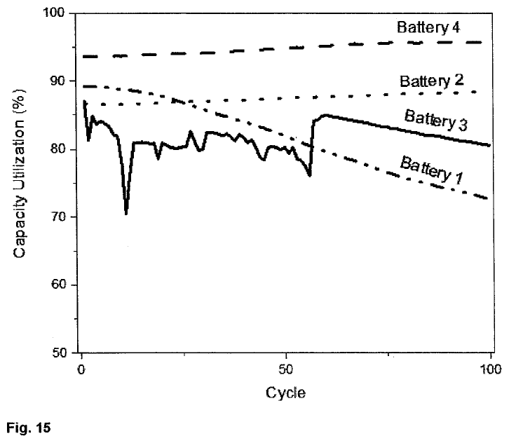

FIG. 15 shows capacity utilization for four different redox flow batteries based on different modified NDIs in combination with different aqueous based electrolytes. The redox flow batteries are for example based on the principle shown in FIG. 12.

The four different redox flow batteries were assembled according to table 4 below. For all four redox flow batteries, the negative electrolyte was based on NDI (denoted NDI-1) or modified NDI (denoted NDI-2) in a potassium chloride (KCl)-potassium phosphate (KPh) solution or NDI-1 and NDI-2 in an ammonium chloride (AmCl)-ammonium phosphate (AmPh) solution, while the positive electrolyte was based on BTMAP-Fc in a KCl-KPh solution and BTMAP-Fc in an AmCl-AmPh solution. NDI-1 and NDI-2 had the general structure according to formula III with the below listed specification:

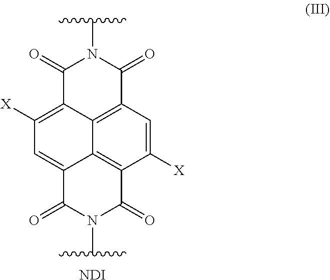

The BTMAP-Fc had a structure according to formula IV:

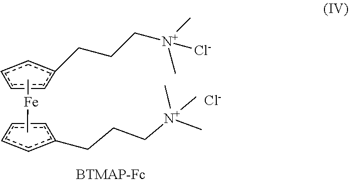

NDI-1 had the specific formula: 2,7-bis(3-(dimethylamino)propyl)benzo[Imn][3,8]phenanthroline-1,3,6,8(2H,7H)-tetraone and NDI-2 had the specific formula: 4,9-bis(dimethylamino)-2,7-bis(3-(dimethylamino)propyl)benzo[Imn][3,8]phenanthroline-1,3,6,8(2H,7H)-tetraone. The first and second redox flow batteries had NDI-1 and the second and third redox flow batteries had NDI-2. The details of the composition of the first, second, third and fourth redox flow batteries are given in table 4 (the negative aqueous-based electrolyte corresponds to the second aqueous-based electrolyte and the positive aqueous-based electrolyte corresponds to the first aqueous-based electrolyte).

TABLE 4

| Redox flow | Negative aqueous- | |
|---|---|---|
| battery no | based electrolyte | Positive Electrolyte |
| 1 | NDI-1 in 1M KCl and | BTMAP-Fc in 1M KCl and |
| | 0.5M KPh | 0.5M KPh |
| 2 | NDI-1 in 1M AmCl and | BTMAP-Fc in 1M AmCl and |
| | 0.5M AmPh | 0.5M AmPh |
| 3 | NDI 2 in 1M KCl and | BTMAP-Fc in 1M KCl and |
| | 0.5M KPh | 0.5M KPh |
| 4 | NDI 2 in 1M AmCl and | BTMAP-Fc in 1M AmCl and |
| | 0.5M AmPh | 0.5M AmPh |

For each one of the four redox flow batteries, 10 ml of the negative electrolyte, and 20 ml of the positive electrolyte was used, to balance the capacities, and the concentration of the redox active materials (BTMAP-Fc, NDI-1 and NDI-2) was 50 mM for all the four redox flow batteries. Each one of the redox flow batteries were cycled (100 cycles) with a current density of 10 mA cm−2, and each cycle took between 1.5 and 2 hours.

The capacity utilizations of the four redox flow batteries shown in FIG. 15 are calculated based on the following relationship:

$$Capacity Utilization = \frac{Discharge Capacity}{Theoretical Capacity} * 100$$

As seen in FIG. 15, a clear trend of diverging capacity utilization factor is seen when comparing the use of ammonium based negative electrolyte (ammonium chloride (AmCl)-ammonium phosphate (AmPh)), i.e. redox flow batteries number 2 and 4 (denoted “Battery 2” and “Battery 4” in FIG. 15), compared to the potassium based negative electrolyte (i.e. potassium chloride (KCl)-potassium phosphate (KPh)), i.e. redox flow batteries number 1 and 3 (denoted “Battery 1” and “Battery 3” in FIG. 15). As a note, the capacity utilization for redox flow battery number 3 fluctuated during the first 60 cycles as a result of a flow obstruction in the electrolyte reservoir. When corrected, the above trend is clearly shown for the following 40 cycles.

In summary, a significant capacity loss is observed for redox flow batteries number 1 and 3 over 100 cycles, while redox flow batteries number 2 and 4 instead show a slow capacity increase. Thus, the impact of the ammonium cation on the cycling stability is apparent for both NDI-1 and NDI-2.

1. A redox flow battery comprising:
a positive compartment comprising a positive electrode in contact with a first aqueous-based electrolyte solution comprising a positive electrolyte dissolved in a first aqueous-based solvent;
a negative compartment comprising a negative electrode in contact with a second aqueous-based electrolyte solution comprising a negative electrolyte being an organic redox-active molecule dissolved in a second aqueous-based solvent;
electrical conductive means for establishing electrical conduction between said positive electrode and said negative electrode, and an external load for directing electrical energy into or out of the redox flow battery;
a separator component that separates the first aqueous-based electrolyte solution in the positive compartment from the second aqueous-based electrolyte solution in the negative compartment and substantially prevents the positive electrolyte in the positive compartment and the negative electrolyte in the negative compartment from intermingling with each other, while permitting the passage of non-redox-active species between the electrolyte solutions in the positive and negative compartments; and means capable of establishing flow of the electrolyte solutions past said positive and negative electrodes, respectively, wherein at least one of the first and second aqueous-based electrolytes is based on an ammonium-based salt, and wherein the organic redox-active molecule is a naphthalene diimide, abbreviated NDI, or a modified NDI.

2. A redox flow battery according to claim 1, wherein the ammonium-based salt comprises at least one of the following: ammonium chloride, ammonium phosphate.

3. A redox flow battery according to claim 1, wherein the other one of the first and second aqueous-based electrolytes is based on an ammonium salt, preferably the same ammonium salt.

4. A redox flow battery according to claim 1, wherein the organic redox-active molecule has a solubility at room temperature of at least 0.4 M in the second aqueous-based electrolyte solution.

5. A redox flow battery according to claim 1, wherein the second aqueous-based electrolyte solution comprises at least two different organic redox-active molecules dissolved in the second aqueous-based solvent, a first organic redox-active molecule being NDI, and a second organic redox-active molecule being modified NDI.

6. A redox flow battery according to claim 1, wherein the modified NDI is a substituted NDI, e.g. a core-aminated NDI.

7. A redox flow battery according to claim 6, wherein the modified NDI comprises an amino group.

8. A redox flow battery according to claim 1, wherein the modified NDI has a structure according to formula I: 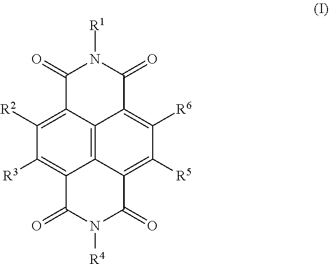
wherein each, of R¹, R², R³, R⁴, R⁵ and R⁶, is independently selected from:
hydrogen atom;
cyano group (—CN);
amino group —NR8R9R10, wherein R10 is present when the amino group is quaternized, and wherein R8, R9 and R10, if R10 present, are independently selected from hydrogen atom and hydrocarbyl group R11 having one to six carbon atoms, or, R8 and R9 forming together with the nitrogen a hetero ring having four to six carbon atoms;
sulfonate (—S(O)₂OH or —S(O)₂O⁻);
halogen atoms (e.g. fluorine, chlorine, bromine, and/or iodine); and
group R7, being a hydrocarbyl group having one to twenty carbon atoms, and having one or more heteroatoms selected from oxygen, nitrogen, sulfur, and halogen atoms (e.g. fluorine, chlorine, bromine, and/or iodine), or
group R7, being a hydrocarbyl group having one to twenty carbon atoms, and being substituted by one, two, or three substituents selected from:
amino group —NR⁸R⁹R¹⁰, wherein R¹⁰ is present when the amino group is quaternized, and wherein R⁸, R⁹ and R¹⁰, if R¹⁰ present, are independently selected from hydrogen atom and hydrocarbyl group R¹¹ having one to six carbon atoms, or R⁸ and R⁹ forming together with the nitrogen, or together with the nitrogen and a further nitrogen in either of R⁸ or R⁹, a hetero ring having four to six carbon atoms, and
sulfonate (—S(O)₂OH or —S(O)₂-O⁻) or (—COOH or or COO—).

9. A redox flow battery according to claim 8, wherein R1 and R4 comprise tertiary or quaternary amines, and/or sulfonate groups.

10. A redox flow battery according to claim 8, wherein at least one of R2, R3, R5 and R6 is a group or molecule comprising more than a hydrogen atom, or is different to a hydrogen atom, or wherein at least one of R2, R3, R5 and R6 is an amino or cyano group.

11. A redox flow battery according to claim 1, wherein the battery is configured such that the NDI, or modified NDI, is reduced with two electrons in the negative compartment, creating an NDI dianion or hydroNDI.

12. A redox flow battery according to claim 11, wherein the NDI dianon or reduced NDI is an original reduced NDI having a first structure, and wherein the battery is configured such that the original reduced NDI is restructured into a restructured reduced NDI having a second structure different from said first structure, the restructured reduced NDI having a different reduction potential compared to the original reduced NDI.

13. A redox flow battery according to claim 12, wherein the difference in reduction potential between the original reduced NDI and the restructured reduced NDI determines the voltage of the battery.

14. A redox flow battery according to claim 1, wherein the pH at the negative compatment is lower compared to the positive compartment with at least a value of pH 2.

15. A redox battery according to claim 1, wherein the pH is adjusted during cycling due to the proton-coupled electron transfer of NDI.

16. A redox flow battery according to claim 1, wherein the positive electrolyte is the same as the negative electrolyte, forming a symmetrical redox flow battery.

17. A redox flow battery according to claim 16, wherein the battery is configured such that the NDI, or modified NDI, is reduced with two electrons in the negative compartment, creating an NDI dianion or hydroNDI, wherein the NDI dianon or reduced NDI is an original reduced NDI having a first structure, and wherein the battery is configured such that the original reduced NDI is restructured into a restructured reduced NDI having a second structure different from said first structure, the restructured reduced NDI having a different reduction potential compared to the original reduced NDI, and wherein charging of the battery results in the following reactions:
in the negative compartment: NDI+2e⁻→NDI₂₋ and NDI₂₋→NDI*²⁻ or NDI+2e⁻+2H⁺→NDIH₂ and NDIH₂→NDIH₂*, wherein NDI*²⁻ and NDIH₂* are forms of the restructured reduced NDI;
in the positive compartment: NDI*2−→NDI*+2e−and NDI*→NDI or NDIH2*→NDI*+2e−+2H+ and NDI*→NDI, wherein NDI* is the oxidised condition of the restructured reduced NDI.

18. An aqueous solution comprising NDI or modified NDI having a structure according to formula II: 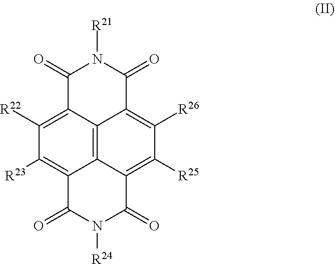
wherein R²² and R²⁵ are both hydrogen atom, and each, of R²¹, R²³, R²⁴ and R²⁶, is independently selected from:
hydrogen atom (H);
cyano group (CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or R²⁸ and R²⁹ forming together with the nitrogen a hetero ring having four to six carbon atoms;
sulfonate (—S(O)₂OH or —S(O)₂O⁻) (—COOH or or COO—);
halogen atoms (e.g. fluorine and/or bromine); and
group R²⁷, being a hydrocarbyl group having one to six carbon atoms, and having one or more heteroatoms selected from nitrogen, sulfur, and halogen atoms (e.g. fluorine and/or bromine), or
group R²⁷, being a hydrocarbyl group having one to six carbon atoms, and being substituted by one or two substituents selected from:
cyano group (—CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or R²⁸ and R²⁹ forming together with the nitrogen, or together with the nitrogen and a further nitrogen in either of R²⁸ or R²⁹, a hetero ring having four to six carbon atoms; and
sulfonate (—S(O)₂OH or —S(O)₂O⁻) or (—COOH or or COO—), wherein the aqueous solution further comprises an aqueous-based electrolyte based on an ammonium-based salt.

19. An aqueous solution according to claim 18, wherein each of R21 and R24, is independently selected from:
group R²⁷, being a hydrocarbyl group having one to six, e.g. one to four, carbon atoms, and having one or more, e.g. one or two, heteroatoms selected from nitrogen, sulfur, and halogen atoms (e.g. fluorine and/or bromine), or
group R²⁷, being a hydrocarbyl group having one to six, e.g. one to four, carbon atoms, and being substituted by one or two, e.g. one, substituents selected from:
cyano group (—CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or R²⁸ and R²⁹ forming together with the nitrogen a hetero ring having four to six carbon atoms; and
sulfonate (—S(O)₂OH or —S(O)₂O⁻), and
wherein R²³ and R²⁶, is independently selected from:
cyano group (CN);
amino group —NR²⁸R²⁹R³⁰, wherein R³⁰ s present when the amino group is quaternized, and wherein R²⁸, R²⁹ and R³⁰, if R³⁰ present, are independently selected from hydrogen atom and hydrocarbyl group R³¹ having one to four carbon atoms, or R²⁸ and R²⁹ forming together with the nitrogen a hetero ring having four to six carbon atoms;
sulfonate (—S(O)₂OH or —S(O)₂O⁻);
halogen atoms (e.g. fluorine and/or bromine); and
group R²⁷, being a hydrocarbyl group having one to six carbon atoms, and having one or more heteroatoms selected from nitrogen, sulfur, and halogen atoms (e.g. fluorine and/or bromine), or
group R²⁷, being a hydrocarbyl group having one to six carbon atoms, and being substituted by one or two substituents selected from:
cyano group (—CN);
amino group —NR²⁸R²⁹R³⁰, wherein R³⁰ is present when the amino group is quaternized, and wherein R²⁸, R²⁹ and R³⁰, if R³⁰ present, are independently selected from hydrogen atom and hydrocarbyl group R³¹ having one to four carbon atoms, or
R²⁸ and R²⁹ forming together with the nitrogen a hetero ring having four to six carbon atoms; and
sulfonate (—S(o)₂OH or —S(O)₂O⁻).
